(19) United States
(12) Patent Application Publication
(10) Pub. No.: US 2024/0412831 A1
(43) Pub. Date: Dec. 12, 2024

(54) MEDICAL DEVICES AND METHODS OF MAKING MEDICAL DEVICES FOR PROVIDING ANNOTATIONS TO DATA

(71) Applicants: Hui Hui, Wuxi, Jiangsu (CN); Dan LI, Shanghai (CN); MengIu JIANG, Shanghai (CN); GE Precision Healthcare LLC, Waukesha, WI (US)

(72) Inventors: Shruti Gadgil, Bangalore (IN); Hui Hui, Wuxi (CN); Dan Li, Shanghai (CN); Menglu Jiang, Shanghai (CN)

(21) Appl. No.: 18/700,596

(22) PCT Filed: Oct. 11, 2021
(86) PCT No.: PCT/CN2021/123107

§ 371 Date: Apr. 11, 2024

(51) Int. Cl. G16H 10/60; G16H 40/67

(52) U.S. Cl. CPC G16H 10/60; G16H 40/67

(57) ABSTRACT

A medical device for a patient including a computing system configured to generate time series data for the patient. A display device is configured to display the time series data generated by the computing system. An annotation module is executable by the computing system and configured to receive an annotation input. The annotation input includes an event time, where the annotation module is configured to time-associate the event time with the time series data and to display a time-based annotation at the event time along with the time series data. The annotation input is provided as at least one of a user input and an automated trigger. A memory system is configured to store the time series data generated by the computing system and the time-based annotation for subsequent display on the display device.

### FIELD

The present disclosure generally relates to medical devices and methods of making and operating medical devices generating time series data.

### BACKGROUND

Patient monitors are essential medical devices, which for example are commonly used within a hospital environment. These and other medical devices generate time series data to provide crucial information for the caregiver to monitor in real-time, and/or to review at a later date. The time series data may include physiological data corresponding to a patient connected to the medical device, and/or other data relating to the functioning of the medical device itself, for example.

Existing platforms for patient monitors include GE Healthcare's® B1×5 M/P patient monitoring device, for example.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One example of the present disclosure generally relates to a medical device for a patient. A computing system of the medical device is configured to generate time series data for the patient. A display device is configured to display the time series data generated by the computing system. An annotation module is executable by the computing system and configured to receive an annotation input, where the annotation input includes an event time, where the annotation module is configured to time-associate the event time with the time series data and to display a time-based annotation at the event time along with the time series data, and where the annotation input is provided as at least one of a user input and an automated trigger. A memory system is configured to store the time series data generated by the computing system and the time-based annotation for subsequent display on the display device.

In certain examples, the time series data is displayed on the display device as one or more waveforms, and the time-based annotation is displayed on the display device as an overlay to the one or more waveforms.

In certain examples, the annotation input is selected from a predefined list of interventions. In further examples, the predetermined list of interventions is limited based on at least one of a model of the medical device and on a user-selected procedure for using the medical device. In further examples, the predefined list of interventions is classified into groups, and where the predefined list of interventions is based on a selection among the groups for display.

In certain examples, the time-based annotation displayed on the display device is selectable to display additional information corresponding to the annotation input.

In certain examples, the computing system is further configured to output the time series data and the time-based annotation to an electronic medical record remote from the medical device.

In certain examples, the time-based annotation is among a plurality of time-based annotations, and wherein the computing system is further configured to generate a list of the plurality of time-based annotations separate from the time series data. In further examples, the computing system is further configured such that selecting one of the plurality of time-based annotations causes the display device to display the one of the plurality of time-based annotations along with the time series data associated therewith.

In certain examples, the computing system is configured to identify group alarm conditions within the time series data, where the annotation module includes trigger criteria for determining whether the group alarm conditions are satisfied, and where the annotation input is automatically provided as the automated trigger when the trigger criteria is satisfied. In further examples, satisfying the trigger criteria requires at least two individual conditions to be satisfied. In further examples, the trigger criteria is user-defined and wherein at least one of the at least two individual conditions relates to physiological data for the patient. In further examples, the annotation input is among a plurality of annotation inputs provided by both the user input and the automated trigger, wherein the user input indicates an intervention in response to the automated trigger, and wherein the computing system is configured to determine a delay between the event times of the user input and the automated trigger and to output the delay to an external device. In further examples, the automated trigger is among a plurality of automated triggers corresponding to a plurality of time-based annotations, and wherein the computing system is further configured to indicate a total of the plurality of automated triggers corresponding to each of the group alarm conditions.

Another example according to the present disclosure generally relates to a method for providing annotations to time series data for a patient from a medical device. The method includes configuring a computing system to generate the time series data for the patient and configuring a display device to display the time series data generated by the computing system. The method further includes providing an annotation module executable by the computing system and configuring the annotation model to receive an annotation input, where the annotation input includes an event time, where the annotation module is configured to time-associate the event time with the time series data and to display a time-based annotation at the event time along with the time series data, and where the annotation input is provided as at least one of a user input and an automated trigger. The method further includes configuring the time series data generated by the computing system and the time-based annotation to be stored in a memory system for subsequent display on the display device. In further examples, the time series data is displayed on the display device as one or more waveforms, wherein the time-based annotation is displayed on the display device as an overlay to the one or more waveforms, and where the annotation module is configured to receive the annotation input as at least one of free text entry and a selection from a predefined list of interventions. In further examples, the predefined list of interventions is classified into groups, and where the predefined list of interventions available for selection is limited based on a selection among the groups. Further examples also include configuring the computing system to identify preset characteristics within the time series data, where the annotation module includes trigger criteria for comparison to the preset characteristics identified, and where the annotation input is automatically provided as the automated trigger when the preset characteristics satisfy the trigger criteria. In further examples, satisfying the trigger criteria requires at least two individual characteristics relating to physiological data for the patient to be among the preset characteristics identified.

Another example according to the present disclosure generally relates to a bedside monitor for a patient. A computing system is configured to generate time series data for the patient. A display device is configured to display the time series data generated by the computing system, where the time series data includes a waveform of physiological data for the patient. An annotation module is executable by the computing system and configured to receive an annotation input, where the annotation input includes an event time, where the annotation module is configured to time-associate the event time with the time series data and to display a time-based annotation at the event time along with the time series data, and where the annotation input is provided as at least one of a user input and an automated trigger. A memory system is configured to store the time series data generated by the computing system and the time-based annotation for subsequent display on the display device. The annotation input is provided as at least one of free text entry, a selection from a predefined list of interventions, and an automated trigger, where the automated trigger is provided when the annotation module identifies preset characteristics within the time series data that satisfies trigger criteria saved in the memory module. The time-based annotation is classified into a group selectable for display on the display device.

Various other features, objects and advantages of the disclosure will be made apparent from the following description taken together with the drawings.

### BRIEF DESCRIPTION OF THE DRAWINGS

The present disclosure is described with reference to the following drawings.

FIG. 1 is perspective view of a medical device according to the present disclosure in-use for monitoring a patient;

FIG. 2 is a schematic view of an exemplary control system such as may be incorporated within the medical device, remote database, and/or external devices;

FIG. 3 is a schematic view of an exemplary data structure and communication scheme for the medical device of FIG. 1;

FIG. 4 is a flow chart of an exemplary method for providing annotations to time series data for a patient according to the present disclosure;

FIG. 5 is an exemplary screenshot of the medical device of FIG. 1 showing the addition of an annotation from a user input according to the present disclosure;

FIG. 6 is an exemplary screenshot of the medical device of FIG. 1 showing the addition of an annotation from an automatic trigger, such as a group alarm, according to the present disclosure;

FIG. 7 is an exemplary screenshot of the medical device of FIG. 6, also showing the creation of a snapshot in conjunction with the automatic trigger according to the present disclosure;

FIG. 8 is an exemplary screenshot of the medical device of FIG. 7 showing a menu for selecting and/or modifying a label for the annotation according to the present disclosure;

FIG. 9 is an exemplary screenshot of the medical device of FIG. 6 showing a menu for selecting applications for execution according to the present disclosure;

FIG. 10 is an exemplary screenshot of the medical device of FIG. 6 showing a graphical view of the annotations according to the present disclosure;

FIG. 11 is an exemplary screenshot of the medical device of FIG. 6 showing a tabular view of the annotations according to the present disclosure;

FIG. 12 is an exemplary screenshot of the medical device of FIG. 11 showing an expansion of one of the annotations in the tabular view according to the present disclosure;

FIG. 13 is an exemplary screenshot of the medical device of FIG. 6 showing a trend view of the annotations according to the present disclosure;

FIG. 14 is an exemplary screenshot of the medical device of FIG. 6 showing a menu for setting up automatic triggers for annotations according to the present disclosure;

FIG. 15 is an exemplary screenshot of the medical device of FIG. 14 showing a menu for setting up individual interventions within groups as automatic triggers according to the present disclosure;

FIG. 16 is an exemplary screenshot of the medical device of FIG. 6 showing a menu for setting up base conditions as automatic triggers according to the present disclosure; and

FIG. 17 is a flow chart of another exemplary method for providing annotations to time series data for a patient according to the present disclosure.

### DETAILED DISCLOSURE

The present inventors have recognized that the medical devices presently known in the art are challenging for caregivers to use while monitoring in real-time, and particularly for subsequent review. In the example of a patient monitoring device as the medical device (e.g., GE Healthcare's® B1×5 M/P patient monitoring device), time series data is generated by the device in real-time and displayed on the display device, for example as a waveform and/or numeric data. This time series data may correspond to ECG, EEG, EMG, heart rate, blood pressure, temperature, and other physiological data, for example, which may be acquired by conventional methods. While this time series data is being generated, different caregivers are providing care in different manners, whether executing a planned procedure (e.g., an operation, monitoring post-operation, etc.), or performing various interventions. In some cases, these interventions are in direct response to the time-series data, for example administering a dose of a drug when a patient's blood pressure or heart rate exceed desirable levels. It should be recognized that the same activity may be characterizable as either an intervention or a procedure, which are generalized terms used only for simplicity unless otherwise stated.

It is common that the data is extensively reviewed after some time has passed, whether by a specialist, intensivist, or other caregivers. The present inventors have recognized that this subsequent analysis of the data is challenging and complicated, requiring not only review of the historical time series data (e.g., the waveforms and numeric data), but also a cross-referencing of any procedures and/or interventions that took place around that time period. In certain examples, the time series data is reviewed by a caregiver on the medical device itself. However, medical devices presently known in the art do not record information relating to procedures and/or interventions, and thus this type of information can only be viewed on a report, or on a remote device, such as a central station connected to the patient's electronic medical record (EMR). This requires the caregiver to memorize various pieces of information while reviewing in the first location (e.g., the EMR), while subsequently reviewing and integrating the collective information at the second location (e.g., the medical device). This practice is time-consuming, limited in the amount of detail that can be considered by the caregiver during analysis, and also leads to human error due to the requirement for memorization and human correlation between the information across several locations. It also limits the responsiveness of a caregiver in delivering immediate care, whereby a caregiver observing a given condition in the current time series data on the medical device cannot immediately react, but must leave to consult the historic information of interventions in the EMR (e.g., by visiting a central reviewing station). In certain examples, the caregiver must also review data from multiple devices when decided to proceed with a given intervention, making subsequent review even harder and time-consuming to track.

As such, the present inventors have developed the presently disclosed medical devices and methods for providing meaningful and concise review of the time series data from the patient along with the history of events corresponding thereto. Additionally, as will be described further below, the disclosed medical devices and methods advantageously provide a mechanism for quickly identifying interventions, procedures, or other events via annotations provided in conjunction with the time series data, viewable together. This allows a caregiver to quickly reference previous landmarks (e.g., how a patient responded to a previous intervention) when deciding additional treatment plans, and also to more quickly review the treatment history (e.g., by an intensivist or supervising physician, for example). Likewise, the presently disclosed medical devices and methods allow the caregiver to filter the time series data to show only that corresponding to the filtered annotations of interest, again reducing the time and effort of finding the desired information.

As will become apparent, certain examples of medical devices and methods disclosed herein further benefit from reducing or eliminating the need for human intervention. This reduces the risk of human error, provides improved case-to-case consistency and compliance, and also improves the efficient workflow of providing care to the patient. Moreover, by reducing or eliminating the need to interact with multiple separate devices (e.g., an EMR in addition to the medical device), the presently disclosed medical devices and methods provide a more robust system in the event of outages or communication failures. For example, a medical device may be outfitted with an uninterrupted power supply such that it may continue functioning during a power outage. However, systems and methods presently known in the art require the caregiver to also be able to interact with a separate system (e.g., and EMR), which may be unavailable during the power outage. Similarly, communication between these devices may be strained in certain contexts, including field applications or in developing regions in which the infrastructure is less stable.

Likewise, the presently disclosed medical devices and methods provide less strain on the communication infrastructure, and also allow for fewer central reviewing locations for caregivers reviewing external systems, such as an EMR.

As stated above, the presently disclosed medical devices and methods advantageously provide time synchronization between annotations and the corresponding, underlying medical data. For manual annotations, the medical device and method provides the user with the flexibility to add this further information at a later point in time if necessary (e.g., if the caregiver is busy performing a procedure). However, the system annotations disclosed herein can in at least some cases provide these annotations automatically (as stated above).

In addition, the present disclosure is configurable to work across a wide variety of medical devices. This advantageously provides for simplicity and uniformity for users in operating differing devices (e.g., a ventilator, an ECG, an anesthesia device, etc.), allows for global rules for automated annotations (e.g., stored in a cloud accessible to the individual medical devices, or for downloading rules locally thereto), and allows for cross-referencing the annotations of multiple medical devices, for example when viewed from a centralized monitoring station or an EMR.

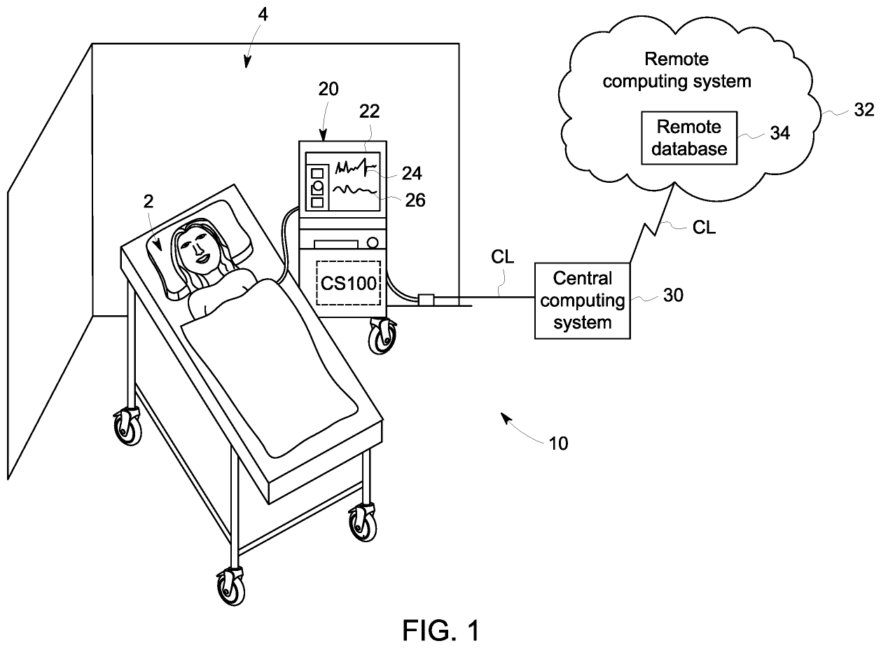

FIG. 1 shows a patient 2 positioned on a bed within a room 4 of a medical facility, such as a hospital or medical clinic. The patient 2 is operatively connected to a medical device 20, such as GE Healthcare's® B1×5 M/P patient monitoring device, such that the caregiver may monitor various physiological parameters in a manner known in the art. The medical device 20 includes a computing system CS100, which controls operation of the medical device 20. Additional information regarding the computing system CS100 is provided below. The medical device 20 shown here further includes a display device 22 that serves as a graphical user interface (for example, a touch-screen GUI 24). Waveforms 26 are produced on the display device 22 for the different physiological parameters being monitored. Additional information may also be provided on the display device 22, which also enables a user to configure the medical device 20 in a customary manner, and also in the manner discussed further below in accordance with the present disclosure.

The medical device 20 is part of a greater system 10, which includes a central computing system 30 operatively connected to the medical device 20 via a communication link CL in a manner presently known in the art. In the configuration shown, the central computing system 30 is further connected to a remote computing system 32, which may be accessible as a cloud computing device over the internet, for example. The remote computing system 32 of the present system 10 further includes, either directly or indirectly, a remote database 34, as discussed further below.

It should be recognized that the central computing system 30 and the remote computing system 32 may be incorporated into a single device, whether positioned locally (e.g., within a hospital) or remotely. Likewise, it should be recognized that the elements of the central computing system 30, the remote computing system 32, and the medical device 20 may be further combined or subdivided from the examples discussed herein while preserving the same function. In certain examples, the central computing system 30 or remote computing system 32 contain or are configured to communicate with the EMR. The medical devices and methods disclosed herein may also be configured to send alerts (e.g., via text message or SMS) when certain automatic annotations are generated, when certain triggers are met, and/or the like to further communicate this information to the primary and/or other caregivers, and quickly.

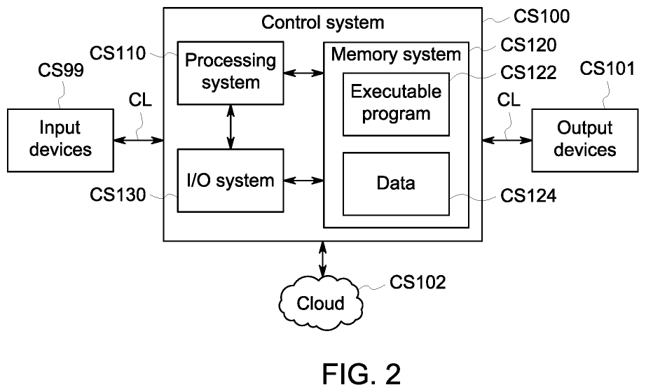

FIG. 2 depicts an exemplary control system CS100 such as may be incorporated within the medical device 20 of FIG. 1. The same of similar structure may also or alternatively be provided as part of the central computing system 30, and/or remote computing system 32 (e.g., with the remote database 34 in certain examples being the memory system CS120). Certain aspects of the present disclosure are described or depicted as functional and/or logical block components or processing steps, which may be performed by any number of hardware, software, and/or firmware components configured to perform the specified functions. For example, certain embodiments employ integrated circuit components, such as memory elements, digital signal processing elements, logic elements, look-up tables, or the like, configured to carry out a variety of functions under the control of one or more processors or other control devices. The connections between functional and logical block components are merely exemplary, which may be direct or indirect, and may follow alternate pathways.

In certain examples, the control system CS100 communicates with each of the one or more components of the system 10 via a communication link CL, which can be any wired or wireless link. The control module CS100 is capable of receiving information and/or controlling one or more operational characteristics of the system 10 and its various sub-systems by sending and receiving control signals via the communication links CL. In one example, the communication link CL is a controller area network (CAN) bus; however, other types of links could be used. It will be recognized that the extent of connections and the communication links CL may in fact be one or more shared connections, or links, among some or all of the components in the system 10. Moreover, the communication link CL lines are meant only to demonstrate that the various control elements are capable of communicating with one another, and do not represent actual wiring connections between the various elements, nor do they represent the only paths of communication between the elements. Additionally, the system 10 may incorporate various types of communication devices and systems, and thus the illustrated communication links CL may in fact represent various different types of wireless and/or wired data communication systems.

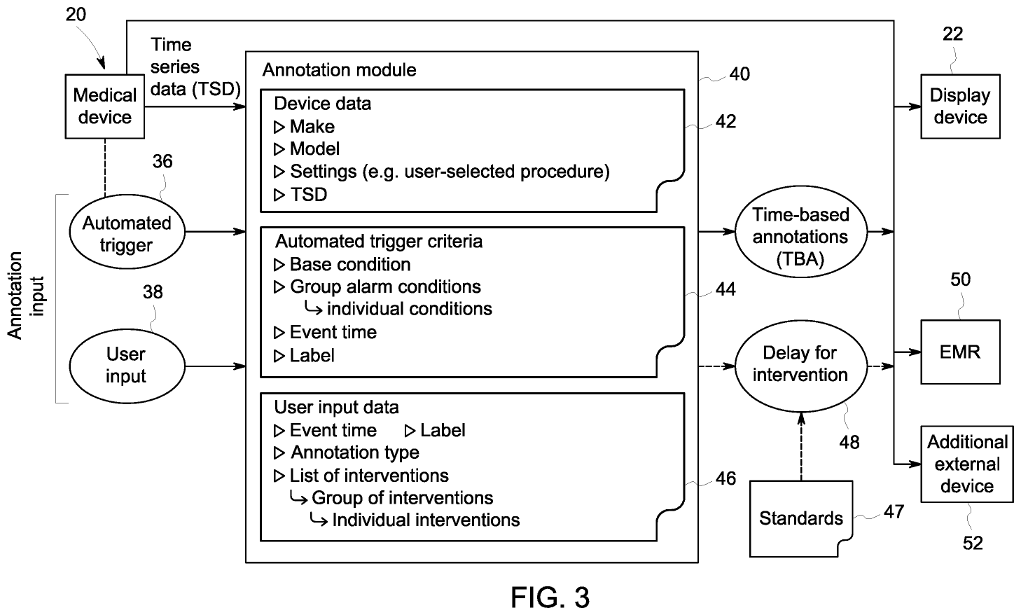

The control system CS100 may be a computing system that includes a processing system CS110, memory system CS120, and input/output (I/O) system CS130 for communicating with other devices, such as input devices CS99 (e.g., sensors and other devices connected to the medical device 20) and output devices CS101 (e.g., the central computing system 30, remote computing system 32, an Electronic Medical Record (EMR 50, see FIG. 3), and/or other external devices (e.g., additional external devices 52 of FIG. 3, such as smart phones or tablets), which may also or alternatively be stored in a cloud CS102). The processing system CS110 loads and executes an executable program CS122 from the memory system CS120, accesses data CS124 stored within the memory system CS120, and directs the system 10 to operate as described in further detail below.

The processing system CS110 may be implemented as a single microprocessor or other circuitry, or be distributed across multiple processing devices or sub-systems that cooperate to execute the executable program CS122 from the memory system CS120. Non-limiting examples of the processing system include general purpose central processing units, application specific processors, and logic devices.

The memory system CS120 may comprise any storage media readable by the processing system CS110 and capable of storing the executable program CS122 and/or data CS124. The memory system CS120 may be implemented as a single storage device, or be distributed across multiple storage devices or sub-systems that cooperate to store computer readable instructions, data structures, program modules, or other data. The memory system CS120 may include volatile and/or non-volatile systems, and may include removable and/or non-removable media implemented in any method or technology for storage of information. The storage media may include non-transitory and/or transitory storage media, including random access memory, read only memory, magnetic discs, optical discs, flash memory, virtual memory, and non-virtual memory, magnetic storage devices, or any other medium which can be used to store information and be accessed by an instruction execution system, for example.

FIG. 3 shows an exemplary data structure for the medical device 20 and an annotation module 40, along with communication flows therebetween. The annotation module 40 may be stored within the memory system CS120 of the medical device 20 and may be comprised of hardware and/or software executable by the processing system CS110. As discussed further below, the annotation module 40 is configured to create annotations and to time-associate the annotations with the time series data generated by the medical device 20. The annotation module 40 is configured to receive an annotation input to generate the annotation, whereby the annotation input is received as a user input 38 (e.g., using a touch-screen or keyboard/mouse associated with the medical device 20), and/or an automated trigger 36. It is contemplated that the annotation input thus could either be received from a user or as an automated trigger from the medical device itself. If the annotation input is from a user, the user inputs 38 may be provided via a touch-screen as the display device 22, and/or using a keyboard and mouse in a customary manner. Automated triggers 36 may be triggered by the medical device 20 itself based on pre-established base conditions and/or preset characteristics (also referred to as “group alarms” being satisfied). Each annotation includes an event time, which enables the annotation to be time-associated with the time series data corresponding thereto appropriately. As is discussed further below, each annotation may also be defined to have a mark (e.g., on the GUI 24), which in certain examples has a shape, size, or color that varies to be easily distinguished from others, and/or a label that is entered as free text, selected from a list of predefined labels, or is automatically provided by the annotation module 40.

FIG. 3 further depicts an exemplary structure for data and logic within the annotation module 40. A medical device data element 42 accesses and/or stores the time-series data (TSD) from the medical device 20, the make and model of the medical device 20 providing the TSD, settings thereof (including a selected procedure for which the medical device 20 is being used, which may include neonatal monitoring, for example). The annotation module 40 further includes an automated trigger criteria element 44, and a user input data element 46, which like the medical device data element 42 may be provided as software, hardware, or combinations thereof.

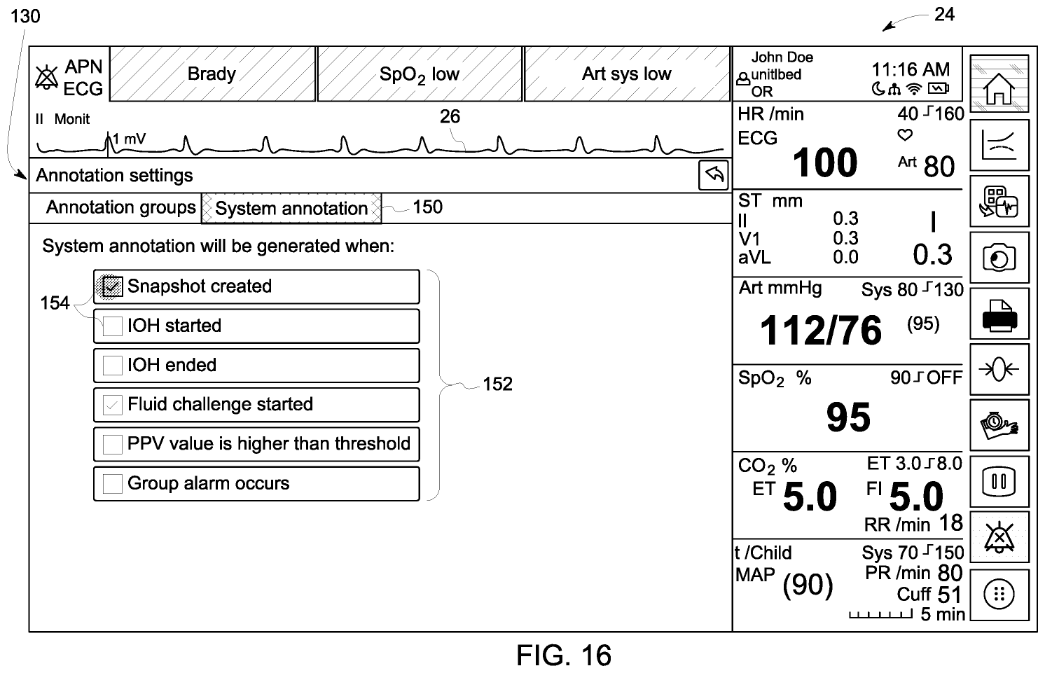

In certain examples, the automated trigger criteria element 44 is a database of base conditions and preset characteristics (also referred to as group alarms) for which the annotation module 40 will automatically generate an annotation when satisfied. Exemplary base conditions include communication failures detected by the medical device 20 (e.g., disconnected ECG leads), physiological data (time series data) outside a threshold range, or other predefined conditions. FIG. 16 depicts an exemplary base conditions menu 150 for selecting base conditions among the individual conditions 152 available, here via check boxes 154. The example shown includes individual conditions 152 relating to a particular application available for execution by the medical device 20, specifically the starting and stopping of an IOH program (intraoperative hypotension application that detects blood pressures falling below a threshold during operations). This enables a caregiver to quickly find within the time series data when the IOH program was running, which often coincides with the data of interest for subsequent review. In the example shown, the base conditions menu 150 also includes a check box 154 for enabling another form of automated trigger, specifically group alarms.

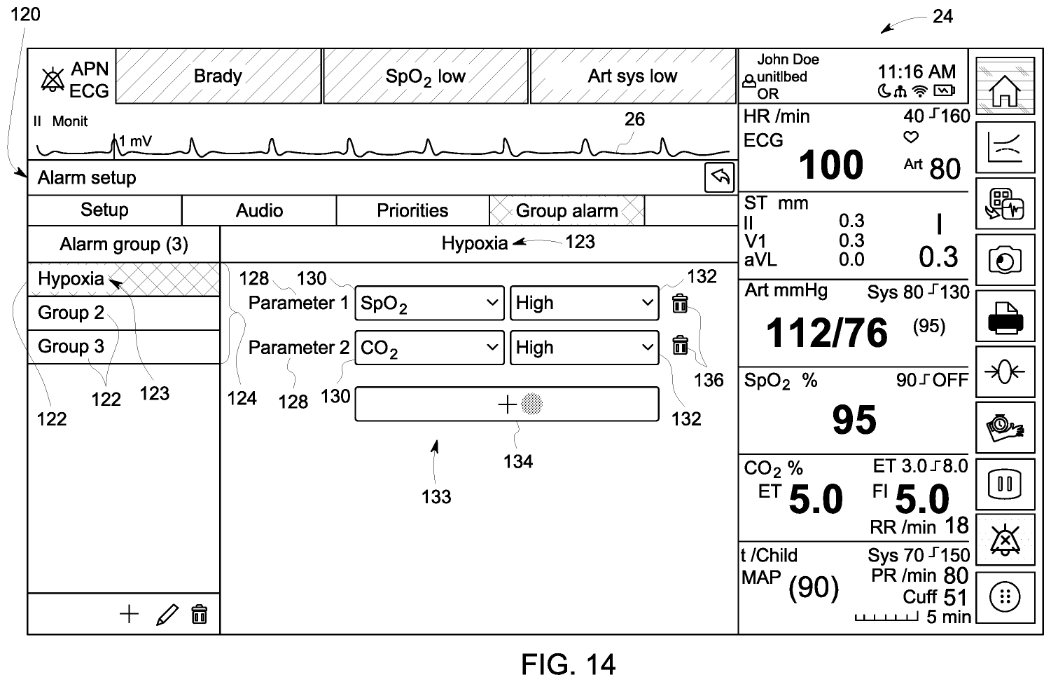

With reference to FIGS. 3 and 14, another type of automatic trigger for which the annotation module 40 automatically creates an annotation is based on the combined state of multiple individual alarms, set up as “group” alarms. In contrast to the base conditions discussed above, group alarms are preset to be triggered when multiple events occur concurrently (satisfying the trigger criteria 133). For example, FIG. 14 shows a group alarm menu 120 having individual groups 122 already created, including a first having a label 123 of “Hypoxia”, which is configured to automatically identify (and annotate) circumstances in which hypoxia is predicted to be occurring. In this example, the trigger criteria 133 for the hypoxia group includes two parameters 128 to be met, here individual parameters 130 corresponding to SpO2 levels and CO2 levels as provided within the time series data of the medical device 20. Specifically, the hypoxia group alarm requires both SpO2 and CO2 to be “high” as the condition 132, meaning that the values of the time series data corresponding thereto exceed a preset threshold. In certain examples, being “high” means that the values exceed the existing alarm limits already set for the medical device 20 in a customary manner. Additional parameters 128 may be added to the group alarm via the add button 134, and likewise, existing parameters 128 deleted via the delete buttons 126. In this manner, the annotation module 40 is configurable to intelligently identify relationships within the time series data, and to flag this identification with an annotation to communicate this fact to the caregiver.

It should be recognized that in certain examples, satisfying the trigger criteria 133 for a group alarm may cause more than the creation of a annotation, such as triggering a local or remote alarm (e.g., at a central monitoring station outside the patient room), creating a record in the EMR, and/or causing a change in the settings or operation of the medical device itself.

In certain examples, the base conditions and/or group alarm conditions (and individual conditions therein) available for selection by the user are limited by the make and model the medical device 20, for example as stored within the medical device data element 42 (FIG. 3). This simplifies the selection process for caregivers by not displaying options that are irrelevant or impossible given the time series data being delivered by the medical device 20.

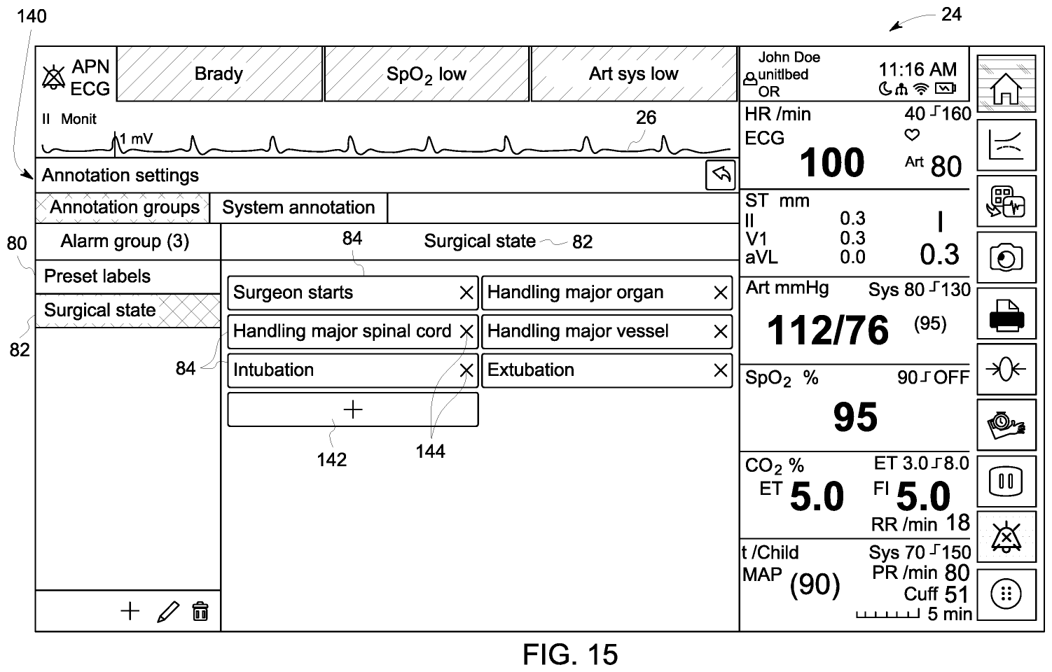

With reference to FIGS. 3 and 15, the annotation module 40 further includes a user input data element 46 for configuring and receiving user input to generate an annotation for association with the time series data. As stated above, the user input can be provided as free text, but can also be selected from among preset labels 80 within the preset selection menu 140 of FIG. 15. In the example shown, two or more of the individual selections 84 among the preset labels 80 can be clustered within a group (also referred to as an annotation type), for example collectively having a label 82 of “surgical states”. Within this example, surgical states includes the individual selections 84 of surgeon starting, handling major spinal cord, major organ, or major vessel, and intubating or extubating. The group of individual selections 84 may also be referred to as a list of interventions, or a series of individual interventions within a group of interventions, for example. This configuration allows a user to quickly filter out the available options for fast selection of an annotation. Additional individual selections 84 may be added to a group via the add button 142, or deleted via the delete button 144.

As shown in FIG. 3, the annotation input provided to the annotation module 40 as either an automated trigger 36 or a user input 38 results in a time-based annotation (TBA) that is time-associated with the time series data (TSD) for later review. In other words, the TBAs are tied to the time series data to coincide in time. This pairing of TBA and TSD may be reviewed on the same display device 22 (e.g., on the medical device 20 itself, rather than requiring departure to review the EMR from a central station), as well as added to the EMR 50 and/or reviewed on additional external devices 52 (e.g., tablets, smart phones, and remote devices).

In certain examples, the present inventors have identified an advantageous configuration in which the responsiveness of a caregiver (e.g., in performing an intervention responsive to an event within the time series data) is compared against a performance standard 47 stored in memory. This may be helpful in quickly performing compliance audits, for example administration of thrombolytic treatment within 180 minutes of the onset of systems or monitoring a 12-lead ECG within a certain time of arriving with chest pain. In these cases, a delay for intervention 48 may be automatically calculated on an ongoing basis and communicated (with or without the corresponding annotation information) to the display device 22, EMR 50, or additional external devices 52.

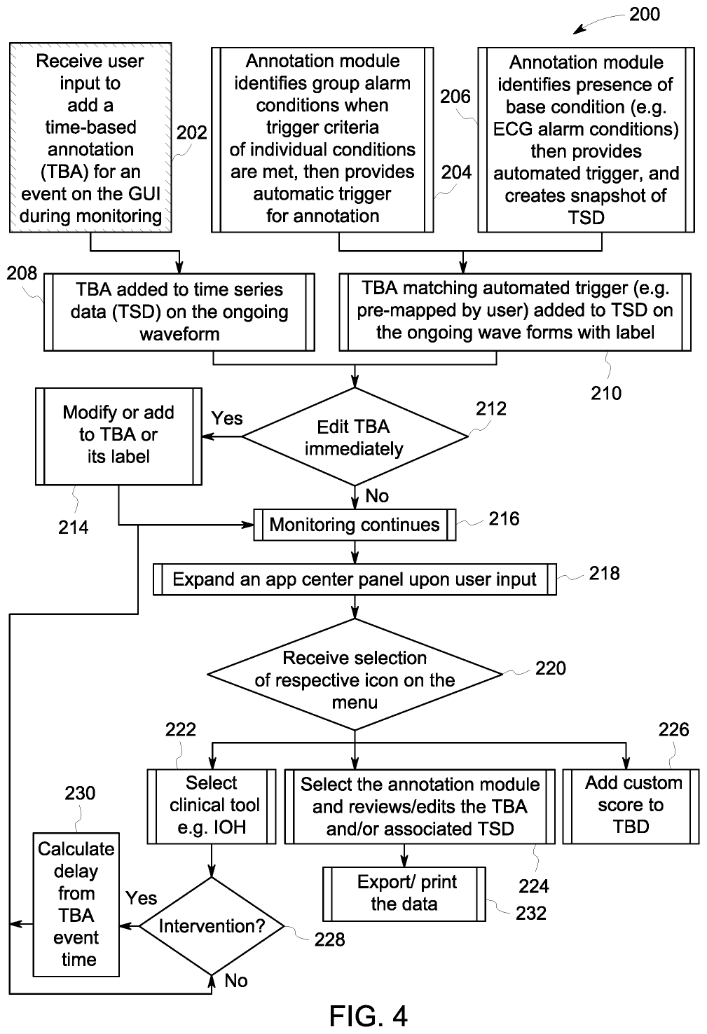

FIG. 4 is a flow chart showing the method 200 for creating annotations according to the present disclosure. As discussed above, the annotation input may arrive as a user input (step 202), or automatic triggers from the medical device as either group alarm conditions (step 204) or base conditions (step 206). The annotation input then results in the time-based annotation (TBA) being added to the time series data (TSD) on the ongoing waveform or waveforms 26 (see FIG. 1), as shown in steps 208 and 210 for the different types of annotation inputs.

From here, the method 200 continues to step 212, whereby the method allows the user to edit the TBA, for example to change or add a label, or to add further comments. If the user decides to edit the TBA, the method proceeds to step 214 for such editing to occur. If the user does not edit the TBA right away, the method proceeds to step 216, whereby the monitoring and/or other functions of the medical device 20 proceed in a customary manner.

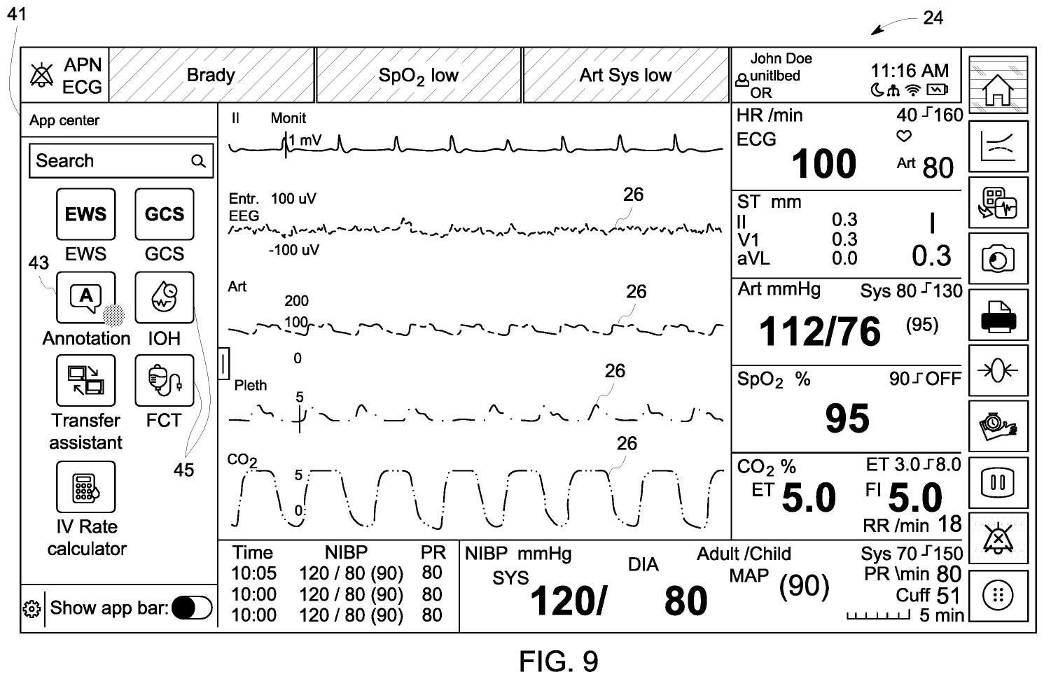

The method allows the user to expand an application center menu 41 (FIG. 9) to later review or edit existing annotations. Specifically, FIG. 9 shows within the application center menu 41 an annotation module icon 43 to access functions of the annotation module 40 (step 220 of FIG. 4), or launch other applications via additional icons 45 (e.g., launching a clinical tool such as the IOH application in step 222). As shown in FIG. 4, if another application is executed in step 222, step 228 determines whether any interventions have occurred, for example the administration of drugs, fluid drips, and/or the like. If no interventions occur, the method returns to step 216 for continued monitoring. However, if an intervention is detected to occur in step 222, the intervention may trigger a further annotation as described above, and/or in the present example a delay between the intervention and the event time of the associated TBA is calculated in step 230 for compliance purposes and the like.

With reference to FIG. 4, if the user selects the annotation module icon 43 presented by the medical device, the method continues to step 224 and/or step 226. In step 224, the method allows the user to select a specific annotation (discussed further below), whereby the label and/or other details may be edited. In step 226, the method allows the user to add a custom score 86 to the annotation to demarcate severity or other criteria for ease of later review. The method allows the user to export and/or print the TBA or the TSD and TBA together in step 232 for subsequent review.

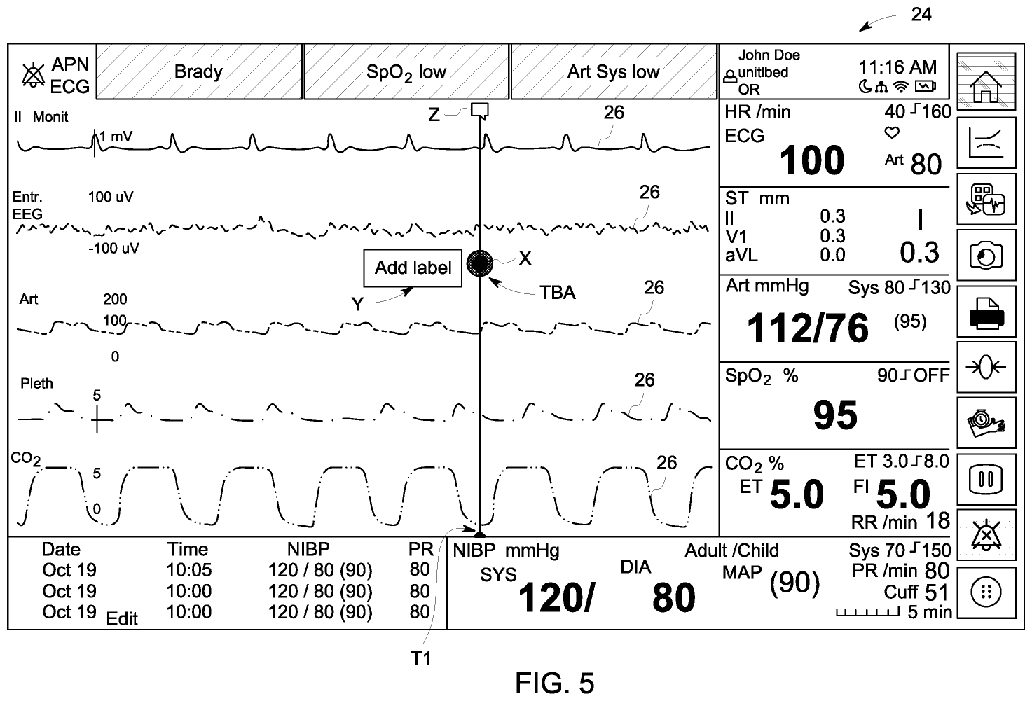

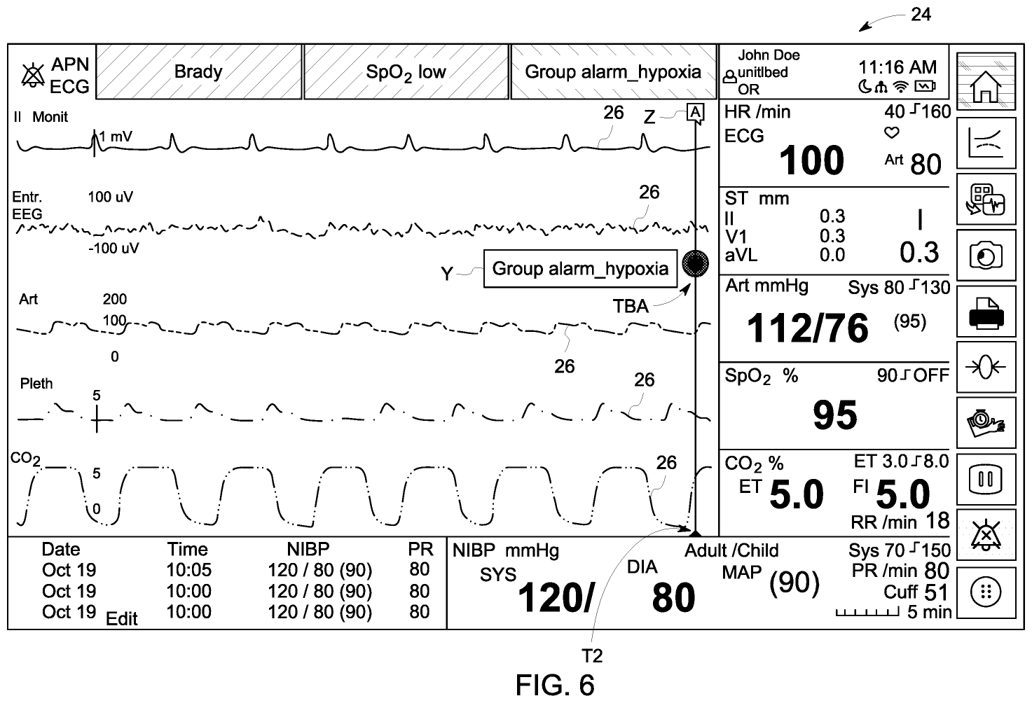

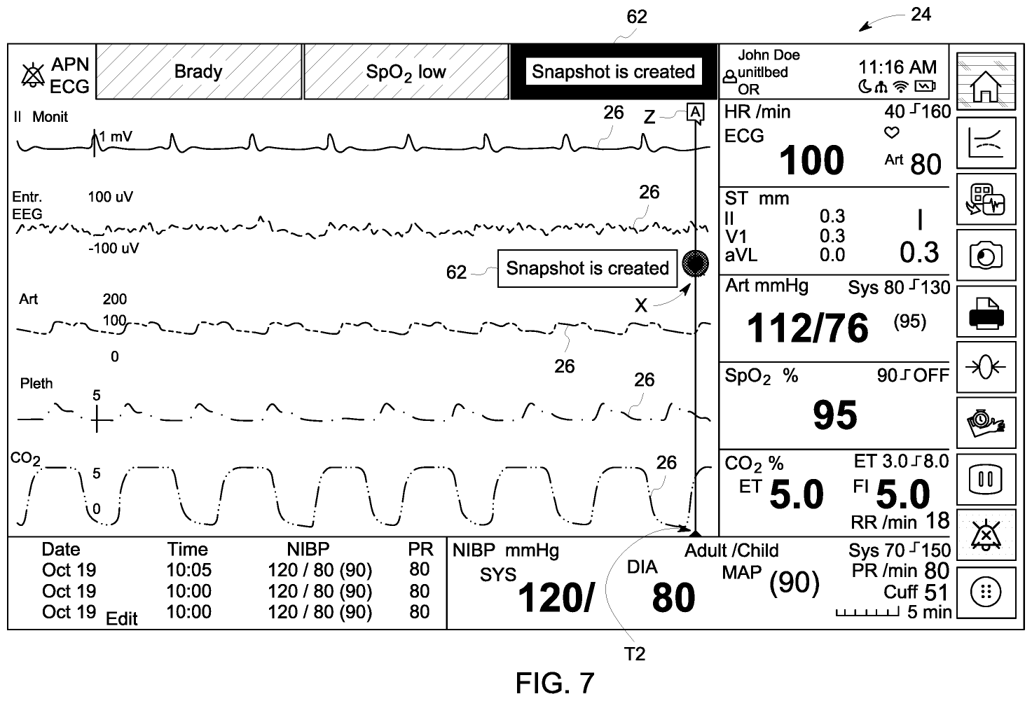

FIG. 5 shows a GUI 24 displaying various waveforms 26 as may be customary for a given medical device 20. However, in addition these waveforms, a time based annotation TBA is displaced at a first time T1 as the event time thereof. In the example shown, the TBA includes a mark X, which may vary in size, color, shape, or other characteristics from other TBAs (or types thereof) such that users can quickly discern between them. The TBA in this example has a placeholder as a label Y, which allows a user to replace this text with a free form message, or to select from among preset labels as discussed above. An additional information icon Z is also provided, whereby when selected, additional information relating to the TBA is shown on the screen. In certain examples, the vertical position of the mark X can also be modified so as to co-locate the TBA with the waveform 26 of interest.

Whereas FIG. 5 showed a TBA generated via a user input as the annotation input, FIGS. 6 and 7 show a TBA generated via an automated trigger as discussed above. In this example, the TBA was automatically triggered as a group alarm for hypoxia, meaning that the individual conditions were met to satisfy the group alarm in automatically triggering the annotation generation, here at second time T2. In certain examples, a snapshot 60 is also created at the same time (second time T2), which may capture relevant time series data collected at that second time T2 for case of review (e.g., in numeric form rather than waveform 26). This snapshot 60 can be viewed by selecting the snapshot icon 62, for example.

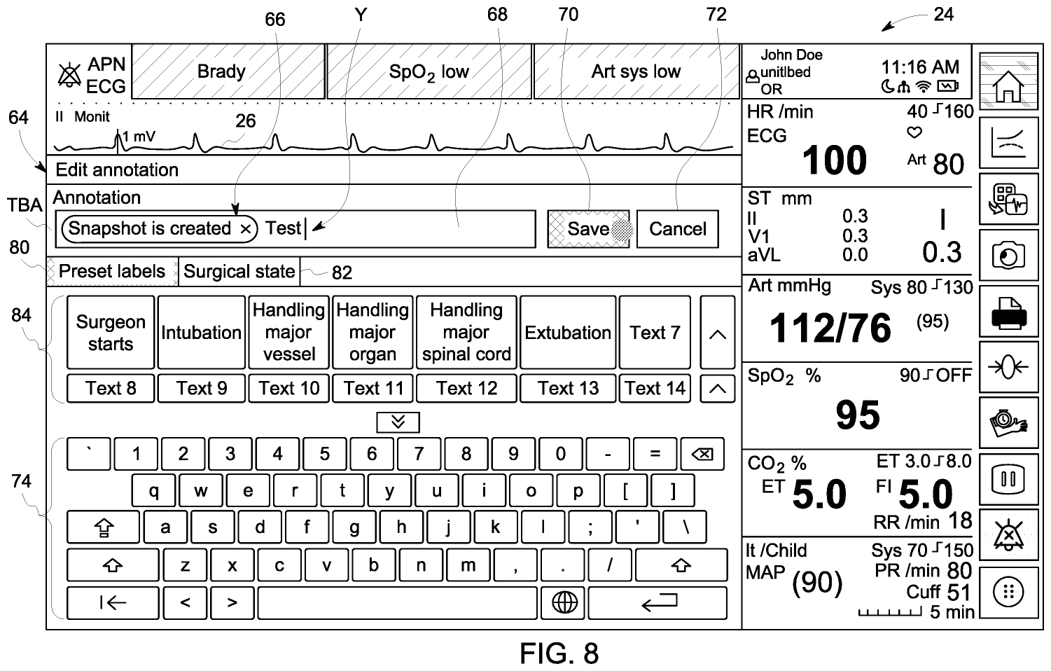

FIG. 8 shows an edit menu 64 that allows a user to edit or add to a given TBA. In the example shown, the edit menu 64 includes preset labels 80 as previously described, which include individual selections 84 available for selection as the label Y for a TBA. The label Y can be changed via label field 68 (e.g., using a virtual keyboard 74) and saved via the save button 70 or cancelled via the cancel button 72.

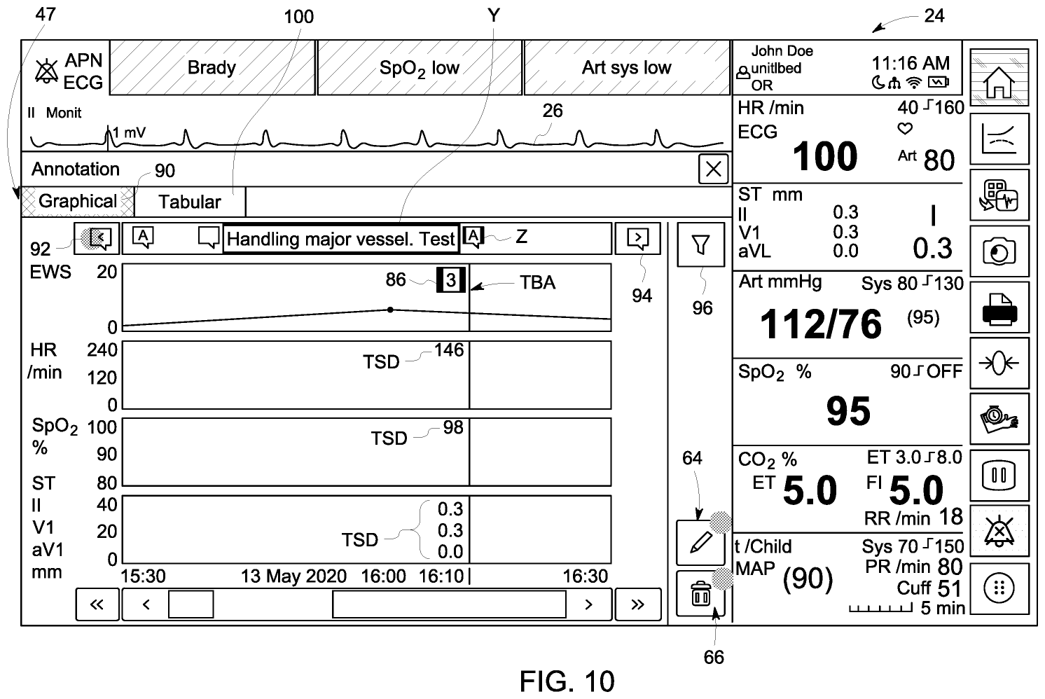

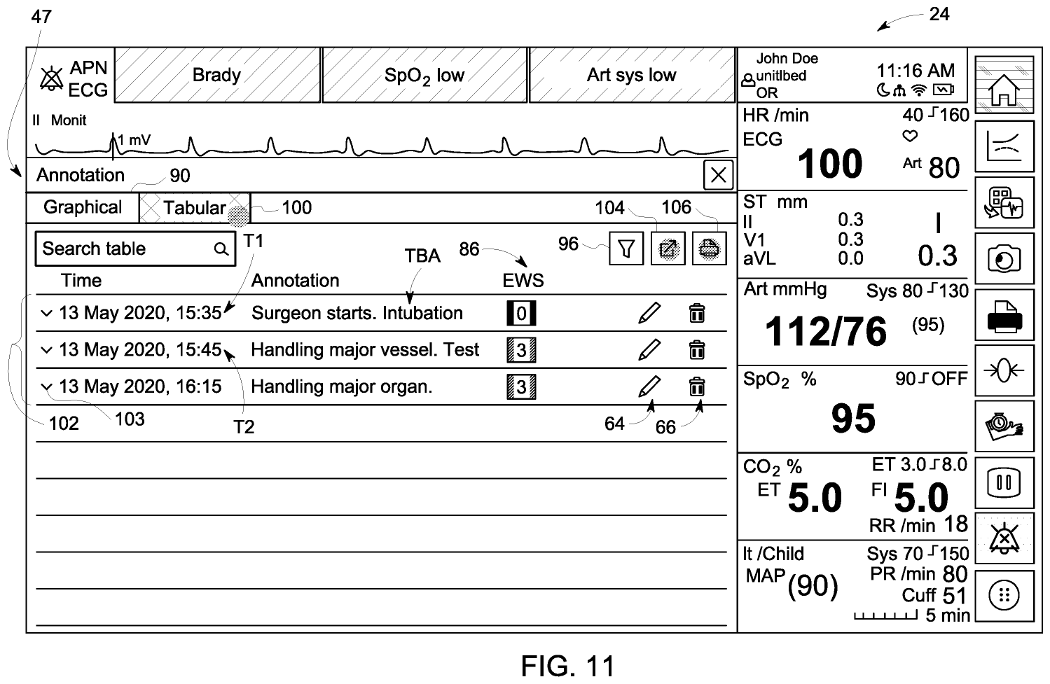

FIGS. 10 and 11 show two ways by which a caregiver may easily review the annotations TBA without scrolling through the time series data on which it is based. FIG. 10 shows a graphical menu 90 that depicts the TBAs over time, along with scores 86 associated therewith. This enables the user to quickly identify critical TBAs over others within the graph of possibilities. Relevant time series data occurring at the event time of each TBA is also shown in the graphical menu 90 to show the caregiver the underlying physiological data collected at that time. The content displayed in the graphical menu may be scrolled back in time via the back arrow 92, or forward via the forward arrow 94.

The present inventors have recognized that it is particularly advantageous to enable the user to filter the TBAs of interest for display, specifically using the filter icon 96. Once the filter icon 96 is selected, a list of available TBAs for selection is provided (similar to filtering rows having particular content in Microsoft Excel®, for example). Unlike devices and methods presently known in the art, this allows a user to quickly view only the relevant portions of a patient's treatment (e.g., only after a particular drug is administered), saving time and avoiding missed patterns or trends by isolating out irrelevant time periods. Filter groups for relevant events may include all surgery related events, all anesthesia related events, all hemodynamic variation related events, and all drug administration related events, for example. The TBA may also be edited via a button for the edit menu 64, or deleted via the delete button 66.

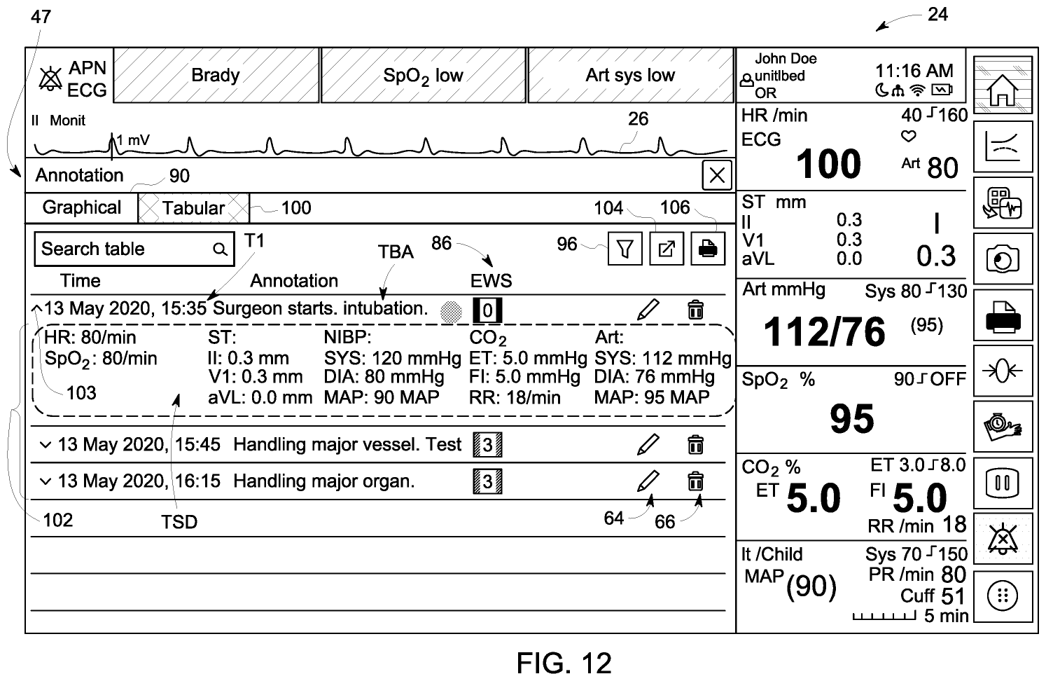

FIG. 11 shows the TBA information as in FIG. 10, but now does so as a list in a tabular menu 100. In this format, each TBA is provided as an individual entry 102 in its own row. Selecting an expand button 103 causes the individual entry 102 to expand as shown in FIG. 12, revealing additional information. In the example of FIG. 12, the additional information includes relevant time series data collected at the event time associated with the TBA, providing useful insights into the actual physiological data behind the annotation TBA.

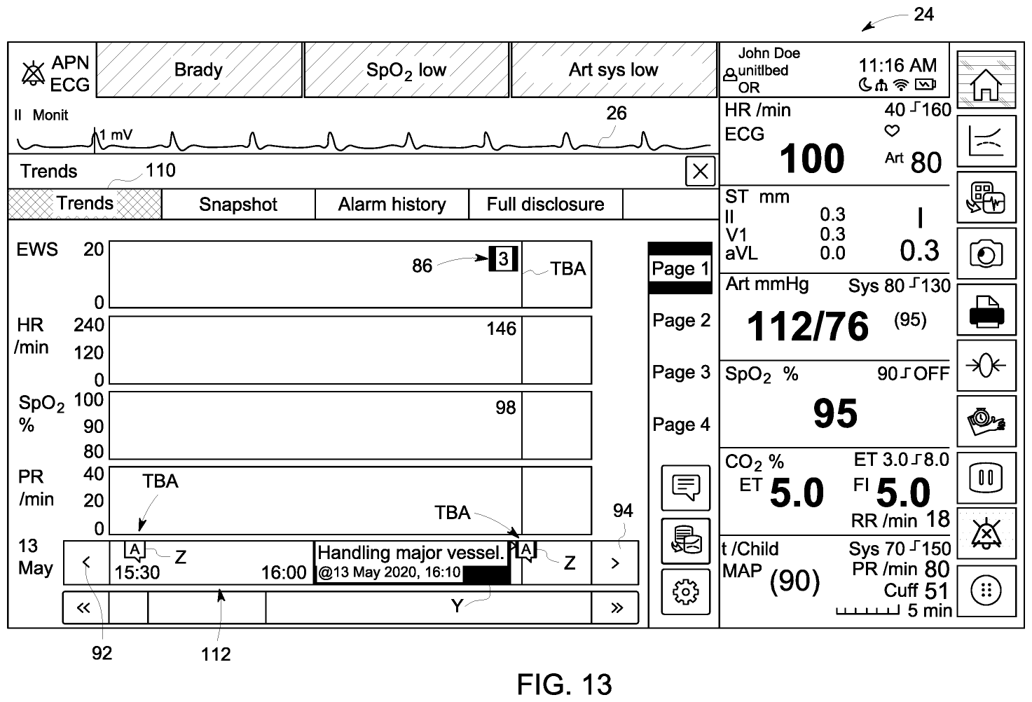

FIG. 13 shows yet another view of the TBA information collected according to the present disclosure, now in trend form within a trending menu 110. Trends can be viewed in the numerical or graphical formats with customized views of the groups defined above, for example.

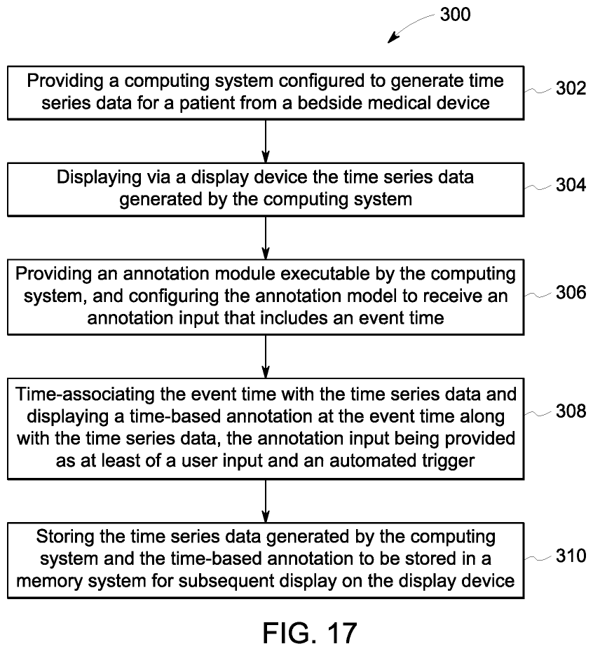

FIG. 17 provides another example of a method 300 for annotating time series data according to the present disclosure. Step 302 provides for providing a computing system configured to generate the time series data for a patient from a medical device. Step 304 provides for displaying via a display device the time series data generated by the computing system, and step 306 for providing an annotation module executable by the computing system and configuring the annotation model to receive an annotation input that includes an event time. In step 308, the annotation module time-associates the event time with the time series data and displays a time-based annotation at the event time along with the time series data, where the annotation input is provided as at least one of a user input and an automated trigger. Step 310 provides for configuring the time series data generated by the computing system and the time-based annotation to be stored in a memory system for subsequent display on the display device.

In this manner, the presently disclosed medical devices and methods provide for fast (and in certain cases automatic) creation of annotations in conjunction with time series data, yielding more concise and meaningful review for a caregiver upon subsequent review. The present inventors have further identified that the quality and consistency of having key events annotated by the immediate caregiver at the time of occurrence is much improved over relying on a subsequent reviewer to identify the same event. This not only saves time, but also reduces human error and improves the effectiveness of treatment plans.

The functional block diagrams, operational sequences, and flow diagrams provided in the Figures are representative of exemplary architectures, environments, and methodologies for performing novel aspects of the disclosure. While, for purposes of simplicity of explanation, the methodologies included herein may be in the form of a functional diagram, operational sequence, or flow diagram, and may be described as a series of acts, it is to be understood and appreciated that the methodologies are not limited by the order of acts, as some acts may, in accordance therewith, occur in a different order and/or concurrently with other acts from that shown and described herein. For example, those skilled in the art will understand and appreciate that a methodology can alternatively be represented as a series of interrelated states or events, such as in a state diagram. Moreover, not all acts illustrated in a methodology may be required for a novel implementation.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A medical device for a patient, the medical device comprising:
a computing system configured to generate time series data for the patient;
a display device configured to display the time series data generated by the computing system;
an annotation module executable by the computing system and configured to receive an annotation input, wherein the annotation input includes an event time, wherein the annotation module is configured to time-associate the event time with the time series data and to display a time-based annotation at the event time along with the time series data, and wherein the annotation input is provided as at least one of a user input and an automated trigger; and
a memory system configured to store the time series data generated by the computing system and the time-based annotation for subsequent display on the display device.

2. The medical device according to claim 1, wherein the time series data is displayed on the display device as one or more waveforms, and wherein the time-based annotation is displayed on the display device as an overlay to the one or more waveforms.

3. The medical device according to claim 1, wherein the annotation input is selected from a predefined list of interventions.

4. The medical device according to claim 3, wherein the predetermined list of interventions is limited based on at least one of a model of the medical device and on a user-selected procedure for using the medical device.

5. The medical device according to claim 4, wherein the predefined list of interventions is classified into groups, and wherein the predefined list of interventions is based on a selection among the groups for display.

6. The medical device according to claim 1, wherein the time-based annotation displayed on the display device is selectable to display additional information corresponding to the annotation input.

7. The medical device according to claim 1, wherein the computing system is further configured to output the time series data and the time-based annotation to an electronic medical record remote from the medical device.

8. The medical device according to claim 1, wherein the time-based annotation is among a plurality of time-based annotations, and wherein the computing system is further configured to generate a list of the plurality of time-based annotations separate from the time series data.

9. The medical device according to claim 8, wherein the computing system is further configured such that selecting one of the plurality of time-based annotations causes the display device to display the one of the plurality of time-based annotations along with the time series data associated therewith.

10. The medical device according to claim 1, wherein the computing system is configured to identify group alarm conditions within the time series data, wherein the annotation module includes trigger criteria for determining whether the group alarm conditions are satisfied, and wherein the annotation input is automatically provided as the automated trigger when the trigger criteria is satisfied.

11. The medical device according to claim 10, wherein satisfying the trigger criteria requires at least two individual conditions to be satisfied.

12. The medical device according to claim 11, wherein the trigger criteria is user-defined and wherein at least one of the at least two individual conditions relates to physiological data for the patient.

13. The medical device according to claim 12, wherein the annotation input is among a plurality of annotation inputs provided by both the user input and the automated trigger, wherein the user input indicates an intervention in response to the automated trigger, and wherein the computing system is configured to determine a delay between the event times of the user input and the automated trigger and to output the delay to an external device.

14. The medical device according to claim 10, wherein the automated trigger is among a plurality of automated triggers corresponding to a plurality of time-based annotations, and wherein the computing system is further configured to indicate a total of the plurality of automated triggers corresponding to each of the group alarm conditions.

15. A method for providing annotations to time series data for a patient from a medical device, the method comprising:
configuring a computing system to generate the time series data for the patient;
configuring a display device to display the time series data generated by the computing system;
providing an annotation module executable by the computing system and configuring the annotation module to receive an annotation input, wherein the annotation input includes an event time, wherein the annotation module is configured to time-associate the event time with the time series data and to display a time-based annotation at the event time along with the time series data, and wherein the annotation input is provided as at least one of a user input and an automated trigger; and
configuring the time series data generated by the computing system and the time-based annotation to be stored in a memory system for subsequent display on the display device.

16. The method according to claim 15, wherein the time series data is displayed on the display device as a waveform, wherein the time-based annotation is displayed on the display device as an overlay to the waveform, and wherein the annotation module is configured to receive the annotation input as at least one of free text entry and a selection from a predefined list of interventions.

17. The method according to claim 16, wherein the predefined list of interventions is classified into groups, and wherein the predefined list of interventions available for selection is limited based on a selection among the groups.

18. The method according to claim 16, further comprising configuring the computing system to identify group alarm conditions within the time series data, wherein the annotation module includes trigger criteria for determining whether the group alarm conditions are satisfied, and wherein the annotation input is automatically provided as the automated trigger when the group alarm conditions are satisfied.

19. The medical device according to claim 10, wherein the trigger criteria is user-defined and wherein at least one of the at least two individual conditions relates to physiological data for the patient.

20. A bedside monitor for a patient, the bedside monitor comprising:
a computing system configured to generate time series data for the patient;
a display device configured to display the time series data generated by the computing system, wherein the time series data includes a waveform of physiological data for the patient;
an annotation module executable by the computing system and configured to receive an annotation input, wherein the annotation input includes an event time, wherein the annotation module is configured to time-associate the event time with the time series data and to display a time-based annotation at the event time along with the time series data, and wherein the annotation input is provided as at least one of a user input and an automated trigger; and
a memory system configured to store the time series data generated by the computing system and the time-based annotation for subsequent display on the display device;
wherein the annotation input is provided as at least one of free text entry, a selection from a predefined list of interventions, and an automated trigger, wherein the automated trigger is provided when the annotation module identifies group alarm conditions within the time series data that satisfy trigger criteria saved in the memory module; and
wherein the time-based annotation is classified into a group selectable for display on the display device.
